(19) United States
(12) Plant Patent Application Publication
(10) Pub. No.: US 2022/0248586 P1
(43) Pub. Date: Aug. 4, 2022

(54) BLUEBERRY PLANT NAMED 'C12-122'

(50) ;

(71) Applicants: COSTA BERRY INTERNATIONAL PTY LTD, Ravenhall (AU); FLORIDA FOUNDATION SEED PRODUCERS, INC., Marianna, FL (US)

(72) Inventors: Gary Wright, Arrawarra Headland (AU); Paul M. Lyrene, Micanopy, FL (US)

(21) Appl. No.: 17/803,052

(22) Filed: Jan. 26, 2022

Related U.S. Application Data
(60) Provisional application No. 63/206,160, filed on Jan. 29, 2021.

(51) Int. Cl. A01H 6/36

(52) U.S. Cl. PLT/157

(57) ABSTRACT

The new blueberry plant variety ‘C12-122’ is provided. ‘C12-122’ is a commercial variety intended for the fresh market. The variety is produced from a cross of unpatented parents ‘FL09-003’ and ‘FL08-013’.

### CROSS-REFERENCE TO RELATED APPLICATIONS

This application claims priority to U.S. Application No. 63/206,160 filed on 29 Jan. 2021, the contents of which are hereby incorporated in their entirety by reference.

Latin name of the family, genus, and species: Family—Ericaceae. Genus—Vaccinium. Species—corymbosum hybrid.

Variety denomination: The new blueberry plant claimed is of the variety denominated ‘C12-122’.

### BACKGROUND OF THE INVENTION

The new variety ‘C12-122’ was selected from a population of seedlings derived from crossing the blueberry varieties known as ‘FL09-003’ (unpatented seed parent) and the variety known as ‘FL08-013’ (unpatented pollen parent). The cross was made in 2009 in Florida, USA and the seed was sown and grown on in Corindi Beach, New South Wales, Australia. The new variety was selected in 2012 from among plants located on land at Corindi Beach and assigned the breeding code ‘C12-122’. Plants of ‘C12-122’ were propagated by cuttings for further evaluation and resulted to be uniform and stable. The new variety showed distinctive traits such as evergreen, vigorous, good size and firm fruit.

### SUMMARY OF THE INVENTION

The new variety ‘C12-122’ was originated from a cross of ‘FL09-003’ (unpatented seed parent) and the variety known as ‘FL08-013’ (unpatented pollen parent) in 2009 in Florida, USA.

The new blueberry variety resulted from seedlings produced in a controlled breeding programme. The cross was made in 2009 in Florida, USA and the seed was sown and grown in Corindi Beach, New South Wales, Australia.

The new variety was selected in 2012 from among plants located on land at Corindi Beach and has since been named ‘C12-122’. Since then plants of ‘C12-122’ were propagated by cuttings for further evaluation and resulted to be uniform and stable. Asexual reproduction of the new variety ‘C12-122’ by cutting propagation since 2012 at Corindi Beach, New South Wales, Australia has demonstrated that the new variety reproduces true to type plants.

The new variety was selected in 2012 as a single plant within a population of seedlings resulting from controlled cross of Vaccinium varieties. The seedling population was planted in an experimental block in the field at Corindi Beach, New South Wales, Australia and the selection of the new variety took place in the same block. Selection criteria were a combination of early season crop, low chilling requirement, strong vigour, non-deciduous type of plant (evergreen) and very large fruit size. The new variety was subsequently evaluated for eight years at the commercial farm at Corindi Beach, New South Wales, Australia.

The following characteristics of the new variety have been repeatedly observed and can be used to distinguish ‘C12-122’ as a new and distinct variety of Vaccinium corymbosum hybrid:
1. Non-deciduous (Evergreen)
2. Early season crop
3. Upright growth habit
4. Low chilling requirement (estimated between 0 and 300 hours)
5. Very large fruit size, average 4.1 g
6. Firm fruit

The new blueberry variety ‘C12-122’ has maintained its distinguished characteristics throughout successive asexual propagation. The variety has been repeatedly asexually reproduced through softwood cuttings in New South Wales, Australia and the clones are phenotypically identical to the original plant.

### BRIEF DESCRIPTION OF THE DRAWINGS

The accompanying photographic illustration shows typical specimens in full color of the foliage and fruit of the new variety ‘C12-122’. The colors are as nearly true as is reasonably possible in a color representation of this type.

FIG. 1 is a photograph of the new variety ‘C12-122’, growing in soil. Photos show 6 year old plants.

FIG. 2 is a photograph of the new variety ‘C12-122’, showing the botanical parts of the plants (blue fruit, green fruit, flowers and leaves).

FIG. 3 is a photograph of the typical fruit cluster of the variety ‘C12-122’.

FIG. 4 is a photograph showing the large fruit size of the new variety ‘C12-122’.

FIG. 5 is a photograph showing the typical fruit calyx of the variety ‘C12-122’.

FIG. 6 is a photograph showing the typical pedicel scar of the fruit of the variety ‘C12-122’.

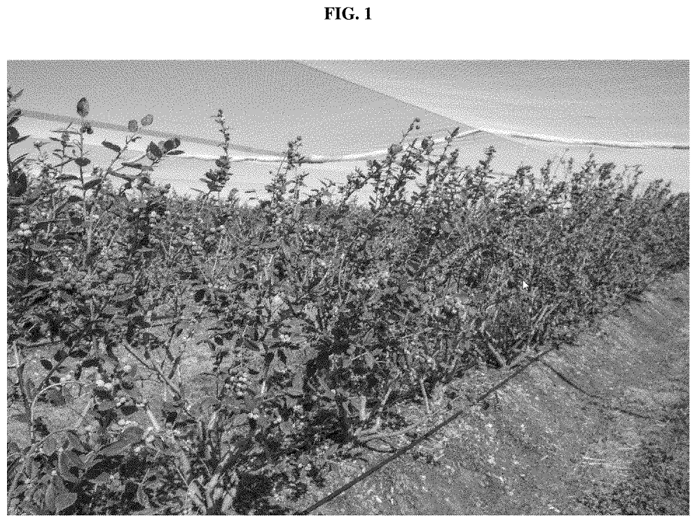

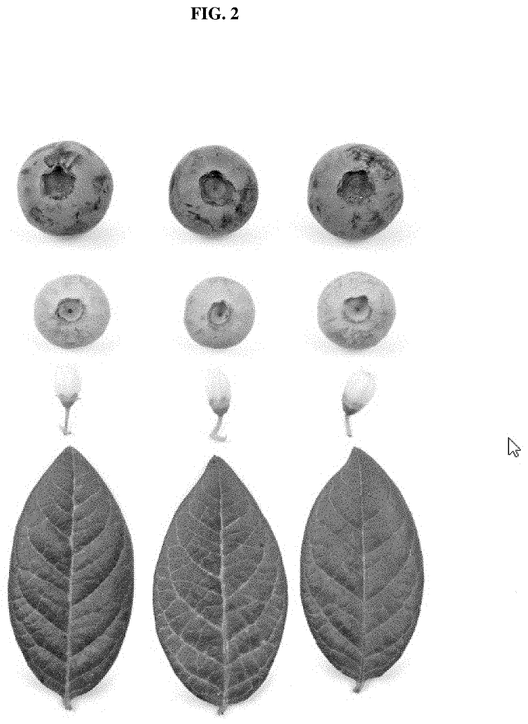

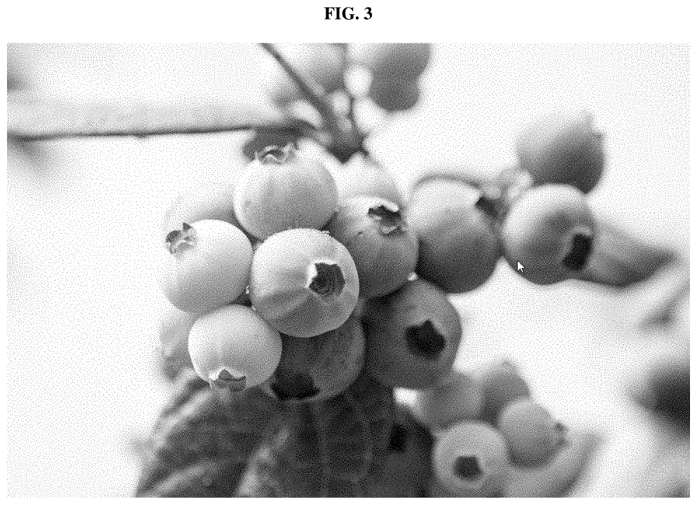

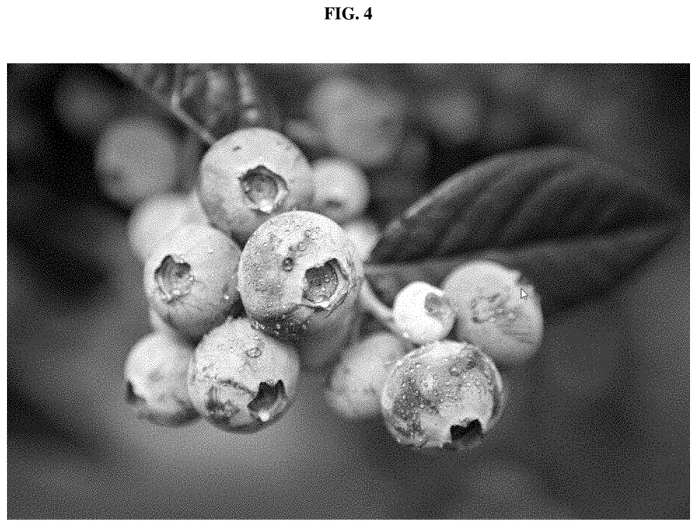

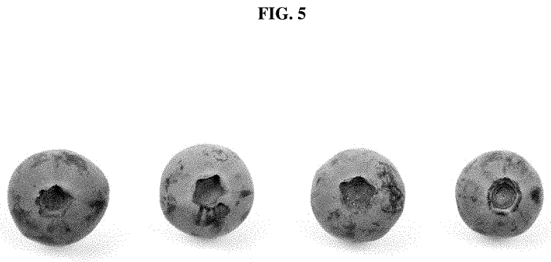

The colors in the photographs are as close as possible with the photographic and printing technology utilized. The color values cited in the detailed botanical description accurately describe the colors of the new blueberry.

### DETAILED BOTANICAL DESCRIPTION

The following detailed description sets forth the distinctive characteristics of ‘C12-122’. The data which defines these characteristics was collected from asexual reproductions of the original selection. Dimensions, sizes, colors, and other characteristics are approximations and averages set forth as accurately as possible. For all traits data was collected from 3 plant parts across 6 randomly selected plants. For the traits relating to fruits (e.g., fruit weight, firmness, brix, acidity) the data is an average across twenty fruits collected randomly. The plant history was taken on plants approximately 3 years of age, and the descriptions relate to plants grown in the field in Corindi Beach, New South Wales, 2456 Australia. Descriptions of fruit characteristics were made on fruit grown in Corindi Beach, New South Wales, 2456 Australia. Color designations are from the 2007 edition of the Royal Horticultural Society (“RHS”).

Classification:
a. Family—Ericaceae.
b. Genus—Vaccinium.
c. Species—corymbosum hybrid.
d. Common Name—blueberry.

Parentage: Female Parent—unpatented selection ‘FL09-003’. Male Parent—unpatented selection ‘FL08-013’.

Market Class: fresh market.

### PLANT

General:
   Parentage.—FL09-003 x FL08-013.
   Plant height.—1.4 m.
   Plant width.—1.1m.
   Growth habit.—upright.
   Growth.—strong vigour.
   Productivity.—high.
   Cold hardiness.—low chill.
   Cold tolerance.—low.
   Chilling requirement.—low, estimated between 0 and 300 hours.
   Tolerance to disease.—none assessed.
   Leafing.—overall medium, plant retains leaves during the winter.
   Twigginess.—low.

### STEM

General:
   Bark texture.—strong.

### FOLIAGE

General:
   Leaf color (top side).—green group, similar to N137B.
   Leaf color (under side).—yellow-green, similar to 147C.
   Leaf arrangement.—alternate.
   Leaf shape.—elliptic.
   Leaf margins.—entire.
   Leaf venation.—reticulate.
   Leaf Length.—long to very long (69.9 mm).
   Leaf width.—broad (36.6 mm).
   Leaf length/width ratio.—1.9.
   Leaf nectarines.—absent.
   Pubescence of upper side.—absent.
   Pubescence of lower side.—absent.
   Attitude.—horizontal.
Petioles:
   Length.—average 3.7 mm.
   Width.—average 2 mm.
   Colour.—yellow-green group 145A.

### FLOWERS

General:
   Time of beginning of flower.—early.
   Flower shape.—urceolate.
   Flower fragrance.—no.
Corolla:
   Color.—white group NN155C.
   Length.—7.3 mm.
   Width.—7.3 mm.
   Aperture width.—2.9 mm.
   Anthocyanin coloration of corolla.—absent or very weak.
   Corolla ridges.—present.
   Protrusion of stigma.—present.
Inflorescence:
   Length.—46 mm.
   Diameter.—31.3 mm.
   Length of peduncle.—35.5 mm.
   Surface texture of peduncle.—medium.
   Color of peduncle.—base colour is yellow-green group 145A, over colour is grey-red group 182A.
   Length of pedicel.—11 mm.
   Surface texture of pedicel.—smooth.
   Color of pedicel.—yellow-green group, similar to 145A and146C.
   Number of flowers per cluster.—10.
   Flower cluster density.—dense.
Stamen:
   Length.—6.3 mm.
   Number per flower.—10.
   Filament color.—yellow-green group 145C.
   Style: Length.—8.15 mm.
   Color.—yellow-green group similar to N144B and 145B.
Pistil:
   Length.—10.3 mm.
Anther:
   Length.—3.5 mm.
   Number.—10.
   Color.—greyed-orange group 165A.
Pollen:
   Abundance.—medium.
   Color.—yellow group 11C.
   Self.—compatibility-yes

### FRUIT

General:
   Time of fruit ripening.—Early
   Cluster density.—dense, 9 berries per cluster.
   Unripe fruit color.—yellow-green-145A.
   Ripe berry color.—blue group 103A.
   Berry surface wax abundance.—strong.
   Berry weight.—very large, average 4.1g.
   Berry height from calyx to scar.—15.6 mm.
   Berry diameter.—20.1 mm.
   Berry shape.—oblate.
   Fruit stem scar.—large and dry.
   Sweetness when ripe.—medium (12 Brix).
   Firmness when ripe.—firm
   Acidity when ripe.—very high (1%).
   Storage quality.—medium to long shelf life (average 24 days).
   Suitability for mechanical harvesting.—not tested.
   Self-fruitfulness.—high, with a large proportion of the crop as parthenocarpic.
   Uses.—fruit to be hand harvested for fresh market.

### SEED

General:
   Seed abundance in fruit.—very low, 1 seed per fruit.
   Seed color.—greyed orange 165A.
   Seed length.—1.7 mm.

### COMPARISON WITH COMMERCIAL CULTIVARS

Table 1 and Table 2 below provide comparisons between ‘C12-122’ and several commercial cultivars:

TABLE 1
Comparison of ‘C12-122’ with similar varieties.

| Characteristics | C12-122 | C99-042 | Snowchaser |
|---|---|---|---|
| Plant vigour | Strong | Weak to | Medium |
| | | medium | |
| Plant growth habit | Upright | Semi-upright to | Semi-upright |
| | | intermediate | |
| One year old shoot | Medium | Very short to | Short |
| length of internodes | | short | |
| Leaf length | Long to very | short | Long |
| | long | | |
| Leaf length (mm) | 69.9 ± 4.7 | 53.10 ± 0.8 | 61.50 ± 0.9 |
| Leaf width | Broad | Very narrow to | Broad |
| | | narrow | |
| Leaf width (mm) | 36.6 ± 2 | 22.9 ± 1.3 | 34.3 ± 1.5 |
| Flower size of corolla | Small | Medium | Medium |
| Flower corolla | 7.3 ± 0.4 | 9.7 ± 0.3 | 9.5 ± 0.3 |
| length (mm) | | | |
| Fruit cluster density | Dense | Sparse | Medium |
| Fruit size | Very large | Small to | Small to |
| | | medium | medium |
| Fruit weight (g) | 4.1 ± 0.9 | 1.9 ± 0.39 | 1.7 ± 0.21 |
| Fruit diameter (mm) | 20.1 ± 1.7 | 15.6 ± 0.96 | 15.10 ± 0.97 |
| Fruit depth of | Medium | Medium | Shallow |
| calyx basin | | | |
| Fruit intensity of | Strong | Weak to | Weak to |
| bloom | | medium | medium |
| Fruit firmness | Firm | Firm | soft |
| Soluble solid | 12 | 13.1 | 14 |
| content (%) | | | |
| Titratable acidity (%) | 1 | 0.4 | 0.7 |
| Time of vegetative | Late | Early | early |
| bud burst | | | |
| Time of beginning | Early | Early to | Very early to |
| of flowering | | medium | early |
| Time of beginning | Early | Early to | Very early to |
| of fruit ripening | | medium | early |

1. A new and distinct variety of blueberry plant named ‘C12-122’, substantially as illustrated and described herein.
